(19) United States
(12) Patent Application Publication
(10) Pub. No.: US 2026/0232923 A1
(43) Pub. Date: Aug. 13, 2026

(54) SUB-ASSEMBLY FOR A MEDICAMENT DELIVERY DEVICE, MEDICAMENT DELIVERY DEVICE, AND METHOD FOR OPERATING A MEDICAMENT DELIVERY DEVICE

(71) Applicant: SHL Medical AG, Zug (CH)

(72) Inventor: Wen-Yen Lee, Taoyuan (TW)

(21) Appl. No.: 19/153,853

(22) PCT Filed: Feb. 5, 2024
(86) PCT No.: PCT/EP2024/052776

§ 371 Date: Aug. 5, 2025

(30) Foreign Application Priority Data
Feb. 9, 2023 (EP) 23155881.8

(51) Int. Cl. A61M 5/32; A61M 5/20; A61M 5/24

(52) U.S. Cl. CPC A61M 5/3257; A61M 5/2033; A61M 5/2422; A61M 2005/2073; A61M 2005/3247

(57) ABSTRACT

The present disclosure provides a sub-assembly for a medicament delivery device. The sub-assembly includes an enclosure, a lock bracket arranged in and rotatable relative to the enclosure, a plunger rod configured to act on a stopper of a cartridge of the medicament delivery device containing a medicament, and a plunger rod spring arranged between the plunger rod and the lock bracket. The lock bracket comprises a mechanical interface portion configured to engage with a corresponding mechanical interface portion of the enclosure for effecting a rotation of the lock bracket relative to the enclosure in response to a force of the biased plunger rod spring acting on the lock bracket in the distal direction. A mechanical switch member of the lock bracket is configured to detect an extended state of the plunger rod relative to the lock bracket along the longitudinal axis; and triggers the rotation of the lock bracket relative to the enclosure.

### TECHNICAL FIELD

The invention refers to a sub-assembly for a medicament delivery device, to a medicament delivery device, and to a method for operating a medicament delivery device.

### BACKGROUND

Medicament delivery devices, in short, delivery devices, include pen-type manual injectors and auto-injectors. These delivery devices are generally known for the self-administration of medicament by patients without formal medical training. For example, patients suffering from diabetes may require repeated injections of insulin, or patients may require regular injections of other types of medicaments, such as growth hormones.

A medicament delivery device receives a medicament cartridge for storing the medicament. The cartridge comprises an outlet at its proximal end and a stopper slidable within the cartridge. The medicament is stored in the cartridge between the outlet and the stopper. In case a medicament delivery is desired, a plunger rod of the delivery device is activated and pushes the stopper further into the cartridge thereby expelling a dose of the medicament at the outlet of the cartridge.

A medicament delivery device additionally comprises an enclosure for the cartridge, and a needle cover sleeve protecting a needle of the delivery device and protecting a user from the needle. The needle cover sleeve is arranged slidable in the enclosure. In a protecting position, the needle cover sleeve protrudes at a proximal end from the enclosure, while during delivery of the medicament, the needle cover sleeve is retracted in the enclosure and exposes the needle.

Once the delivery of the medicament is accomplished and the needle cover sleeve is pushed into its protecting position again, e.g. by a needle cover spring, it is desired that another retraction of the needle cover sleeve and corresponding exposure of the needle is prevented to protect the user and or others in a subsequent handling and/or disposal process.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to a first aspect of the present invention, a sub-assembly is provided for a medicament delivery device. The sub-assembly extends along a longitudinal axis between a proximal end and a distal end. The sub-assembly comprises an enclosure, a lock bracket arranged in and rotatable relative to the enclosure, a plunger rod configured to act on a stopper of a cartridge of the medicament delivery device containing a medicament, and a plunger rod spring arranged between the plunger rod and the lock bracket, preferably for moving the plunger rod and the lock bracket relative to the enclosure. The plunger rod spring is configured to move the plunger rod from a distal position to a proximal position relative to the enclosure. Preferably, the plunger rod is in the proximal position e after delivery of the medicament is accomplished. Alternatively, the proximal position of the plunger rod is primed, e.g., a position where air within the cartridge is expelled. Alternatively, the proximal position of the plunger rod is mixed, e.g., a position in that multiple substances in multiple chambers of the cartridge are mixed.

The lock bracket comprises a mechanical interface portion contributing to a mechanical interface between the lock bracket and the enclosure. Accordingly, the enclosure comprises a corresponding mechanical interface portion. The mechanical interface is configured to effect a rotation of the lock bracket relative to the enclosure in response to an engagement of the respective interface portions and in response to a force exerted by the plunger rod spring in a biased state and pushing the lock bracket in the distal direction.

The lock bracket further comprises a flexible arm extending in the direction of the longitudinal axis along a portion of the plunger rod. The flexible arm comprises a distally directed surface. The flexible arm is configured to flex radially inwardly relative to the enclosure from a first position where the distally directed surface of the flexible arm is engaged with a proximally directed surface of the enclosure to a second position where the distally directed surface of the flexible arm is disengaged with the proximally directed surface of the enclosure.

The plunger rod is radially lined up with the flexible arm when the plunger rod is in the distal position so that the flexible arm is blocked from moving into the second position and spaced apart from the flexible arm when the plunger rod is in the proximal position such that the flexible arm flexes in the second position. In case the enclosure comprises a rear enclosure as will be introduced later on.

Accordingly, when the flexible arm flexes in the second position, the plunger rod spring pushes the lock bracket in the distal direction to interact with the mechanical interface portions and affects the rotation of the lock bracket relative to the enclosure. This rotation of the plunger rod can be used for one or more purposes, such as for indicating an accomplished delivery of the medicament, and/or for locking a needle cover sleeve in a needle-protecting position.

In a preferred embodiment, the mechanical interface portion is arranged at the distal end of the lock bracket and preferably comprises a flange with one or more tooth/teeth protruding in the distal direction, the teeth preferably being arranged circumferentially on the flange. This embodiment results in a compact implementation of the mechanical interface. Preferably, each tooth of the mechanical interface portion comprises a helical surface configured to effect the rotation of the lock bracket when engaged with the corresponding mechanical interface portion of the enclosure.

The enclosure preferably comprises a tubular enclosure including an opening at its distal end and a rear enclosure. The rear enclosure preferably is attached to the tubular enclosure and comprises a cover for covering the opening. Given the mechanical interface portion of the lock bracket preferably facing the cover of the rear enclosure, the corresponding mechanical interface portion of the enclosure is provided at the rear enclosure, and preferably comprises at least one tooth protruding from the cover in the proximal direction. In a preferred embodiment, each tooth of the corresponding mechanical interface portion of the rear enclosure also comprises a helical surface configured to effect the rotation of the lock bracket when engaged with the helical surface of the corresponding tooth of the mechanical interface portion of the lock bracket. Preferably, multiple teeth of the corresponding mechanical interface of the rear enclosure are arranged circumferentially on the cover, preferably at a radius corresponding to the radius of the arrangement of the teeth of the lock bracket's flange. Preferably, the number of teeth of the lock bracket is identical to the number of teeth of the rear enclosure. Accordingly, the rotation of the lock bracket relative to the rear enclosure is firmly guided.

Preferably, the enclosure comprises a protrusion protruding from the cover of the rear enclosure in the proximal direction; wherein the protrusion comprises the proximally directed surface of the enclosure.

Preferably, all members belonging to the lock bracket are made in one piece, i.e. integral. Hence, it is preferred that the flange, the interface member, the mechanical switch member and the bracket retaining member are manufactured in one piece, e.g. made from plastics or metal. The same preferably holds for the rear enclosure: Accordingly, it is preferred that the cover, the guide rod, the corresponding interface portion and the bracket retaining counterpart member are manufactured as one piece, e.g. made from plastics.

In a preferred embodiment, the rear enclosure comprises a guide rod extending in the proximal direction and configured to guide a movement of the plunger rod along the longitudinal axis. The plunger rod spring is radially arranged between the guide rod and the plunger rod. And the lock bracket is slidable and arranged on the guide rod against the force of the plunger rod spring. The transversal flange of the lock bracket preferably contains an opening the guide rod reaches through. The plunger rod preferably is tubular shaped and comprises a front at its proximal end. The plunger rod spring reaches into the tubular plunger rod and is biased between the front of the plunger rod and the flange of the lock bracket.

In a preferred embodiment, an indicator is provided for one or more visually, audibly and tactile indicating an extended state of the plunger rod and/or a locked state of the lock bracket. In a first variant, the lock bracket triggers a signal of one of the visual, audible or tactile properties in response to the plunger rod spring-actuated movement in a distal direction towards the rear enclosure. In a second variant, the lock bracket triggers a signal of one of its visual, audible or tactile properties in response to the lock bracket having terminated its rotation relative to the enclosure and being in a locked state. Such an indicator may support the user in learning when delivery is considered accomplished by the delivery device, and/or when the needle of the delivery device is protected.

According to another aspect of the present invention, a medicament delivery device is provided comprising a sub-assembly according to any of the preceding embodiments, a needle to expel medicament from a cartridge of the medicament delivery device, and a needle cover sleeve arranged in and slidable relative to the enclosure between a needle protecting position and a needle exposing position. Preferably, a needle cover spring is provided for driving the needle cover sleeve in the proximal direction relative to the enclosure.

Preferably, the lock bracket comprises a needle cover sleeve locking member configured to interact with the needle cover sleeve in response to the rotation of the lock bracket. The needle cover sleeve locking member is the part of the lock bracket that after rotation of the lock bracket prevents the needle cover sleeve from further movement along the longitudinal axis and relative to the enclosure, and in particular in the distal direction thereby preventing from exposing the needle at the proximal end of the delivery device. Accordingly, in the engaged position of the needle cover locking member and the needle cover sleeve, a transition of the needle cover sleeve from the needle protecting position into the needle exposing position is prevented. This protects the user of the delivery device or any other person, e.g. being responsible for the disposal of the delivery device, from being injured or even infected by an exposed needle.

Preferably, the needle cover sleeve locking member comprises one or more legs extending from the flange of the lock bracket into the proximal direction.

At the distal end of the needle cover sleeve, a protrusion is preferably arranged to engage with the needle cover sleeve locking member in response to the rotation of the lock bracket. Preferably, this protrusion is arranged radially inwards from an inner surface of the tubular needle cover sleeve. Such protrusion preferably comprises a transversal locking surface that engages with a transversal locking surface of the needle cover sleeve locking member.

Accordingly, after the detection of an accomplished delivery of the medicament, the lock bracket is transitioned, by way of rotation, into a position and a locking state in which the lock bracket prevents the needle cover sleeve from being withdrawn from its needle-protecting position.

In an embodiment of the present invention, the delivery device comprises a cartridge drive of a tubular-shaped that is coupled at its proximal end with a distal end of a cartridge holder. The cartridge holder preferably holds a cartridge with the medicament. The cartridge preferably comprises a stopper arranged slidable in the tubular-shaped cartridge. The plunger rod, driven by the plunger rod spring in the proximal direction, pushes the stopper further into the cartridge and causes the medicament to expel at the proximal end of the cartridge.

Preferably, an actuator such as the biased needle cover spring pushes the cartridge driver in the proximal direction relative to the enclosure. Accordingly, the cartridge driver is arranged slidable within the delivery device, in particular for driving a cartridge held by the cartridge holder towards a stationary needle hub supporting the needle to penetrate a seal of the cartridge by the needle in response to this relative movement.

According to a further aspect of the present invention, a method is provided for operating a medicament delivery device according to any of the above embodiments. The medicament delivery device is provided in a non-extended state of the plunger rod relative to the lock bracket.

In a preferred embodiment, in response to the needle cover sleeve being pushed by a needle cover spring from the needle exposing position, e.g. after medicament delivery is accomplished, into the needle protecting position, the rotation of the lock bracket with respect to the enclosure is finalized, in which final position of the lock bracket after rotation the needle cover sleeve is locked from being pushed back into the enclosure by the lock bracket.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings, wherein

FIG. 1 illustrates a partially transparent perspective view of a medicament delivery device in an initial state, according to an embodiment of the present invention;

FIG. 2 illustrates a partially transparent perspective view of a medicament delivery device in a prepared state, according to an embodiment of the present invention;

FIG. 3 illustrates a partially transparent perspective view of a medicament delivery device in a state during delivery, according to an embodiment of the present invention;

FIG. 4 illustrates a perspective view of the components of the medicament delivery device in a state of accomplished delivery;

FIG. 5 illustrates an exploded view of a medicament delivery device according to an embodiment of the present invention;

FIG. 6 illustrates a lock bracket as used in a sub-assembly according to an embodiment of the present invention, in two perspectives a) and b);

FIG. 7 illustrates a rear enclosure as used in a sub-assembly according to an embodiment of the present invention;

FIG. 8 illustrates a sub-assembly in an initial state, according to an embodiment of the present invention;

FIG. 9 illustrates a sub-assembly in a state during delivery, according to an embodiment of the present invention;

FIG. 10 illustrates a sub-assembly in a state of accomplished delivery, according to an embodiment of the present invention;

FIG. 11 illustrates the sub-assembly of FIG. 10 including a needle cover sleeve 7;

FIG. 12 illustrates the sub-assembly of FIGS. 10 and 11 in a locked state;

FIG. 13 illustrates the sub-assembly of FIG. 12 from a different perspective.

### DETAILED DESCRIPTION

The same components and elements are referred to by the same reference signs across all Figures.

FIG. 1 illustrates a partially transparent perspective view of a medicament delivery device 100 in an initial state, according to an embodiment of the present invention.

The delivery device 100 extends along a longitudinal axis illustrated by a dashed arrow between a proximal end P and a distal end D. The delivery device 100 presently takes an initial state, i.e. a state in which the delivery device 100 may be distributed to recipients.

The delivery device 100 comprises a tubular-shaped enclosure 8 illustrated transparently and a cover-shaped rear enclosure 16 for closing a distal opening of the tubular enclosure 8. Tubular enclosure 8 and rear enclosure 16 are collectively referred to as enclosure 8,16. The tubular enclosure 8 encloses a needle cover sleeve comprising two tubular-shaped portions 6 and 7, preferably of the same diameter and circumference, coupled to each other. A plug 5 is attached to the proximal end of the needle cover sleeve 6,7, and a cap 4 completes the delivery device 100 at its proximal end P.

A cartridge driver spring 12 is arranged between the needle cover sleeve 6,7 and the rear enclosure 16 and is biased in an initial state of the delivery device. Accordingly, given that the rear enclosure 16 is firmly attached to the tubular enclosure 8, the cartridge driver spring 12 pushes the needle cover sleeve 6,7 in the proximal direction.

The needle cover sleeve 6,7 encloses, in the initial state, a combination of a cartridge 10 held by a cartridge holder 9 and a cartridge driver 11. At the distal end D, a plunger rod 13 is drivable by a biased plunger rod spring 14. The plunger rod 13 reaches into and through the tubular-shaped cartridge driver 11 and, during delivery of the medicament, acts on a stopper of the cartridge 10.

A mechanical interface is provided between the cartridge holder 9 and the tubular enclosure 8. The mechanical interface presently encompasses an interface member 91 of the cartridge holder 9 shaped as a protrusion, and an interface member 81 of the tubular enclosure 8 in form of a stair structure protruding from an inner surface of the tubular enclosure 8. In the present initial state, the interface member 91 is locked in a first position relative to the tubular enclosure 8, in a first step of the stair structure of the interface member 81. The step prevents the cartridge holder 11 from being driven towards the proximal end under the impact of a force exerted by the biased cartridge driver spring 12 pushing on the cartridge driver 11, which cartridge driver 11 is coupled with the cartridge holder 9.

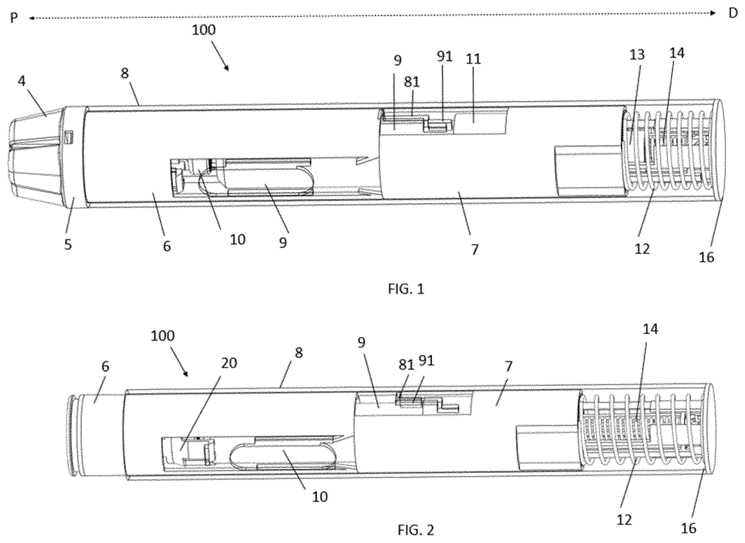

FIG. 2 illustrates a partially transparent perspective view of a medicament delivery device 100 in a prepared state, according to an embodiment of the present invention, the delivery device preferably being the delivery device of FIG. 1. In response to a preferably manual rotation of the cap 4 that is rotatably coupled with the cartridge holder 9, the cartridge holder 9 is released from its initial first, distal position relative to the tubular enclosure 8. As a result, the cartridge 10, the cartridge holder 9 and the cartridge driver 11 are pushed in the proximal direction by the cartridge driver spring 12 towards a needle hub 20 arranged stationary in the tubular enclosure 8 and comprising a needle facing the cartridge 10, and in particular a seal of the cartridge 10. Finally, the needle of the needle hub 20 penetrates the seal of the cartridge 10 and couples the needle arranged in the delivery device with the cartridge being provided absent an own needle and inserted into the cartridge holder 9.

Given that the cartridge driver spring 12 also acts on the needle cover sleeve 6,7, the needle cover sleeve 6,7, presently portion 6, protrudes from a proximal end of the tubular enclosure 8. In such protruded position, the needle cover sleeve 6,7 protects the needle. Preferably, a mechanism is provided to also prime the cartridge 10 in this state, i.e., remove residual air from the cartridge, preferably by automatically making the plunger rod 16 push a stopper of the cartridge 10 for a distance into the cartridge 10 sufficient to expel air from the cartridge 10, but insufficient to expel the medicament out of the cartridge 10. The present state with the needle cover sleeve protruding from the enclosure, the cartridge being primed, and the cap being removed is also referred to as a prepared state of the delivery device, since now the delivery device is ready to deliver the medicament in response to a user's action.

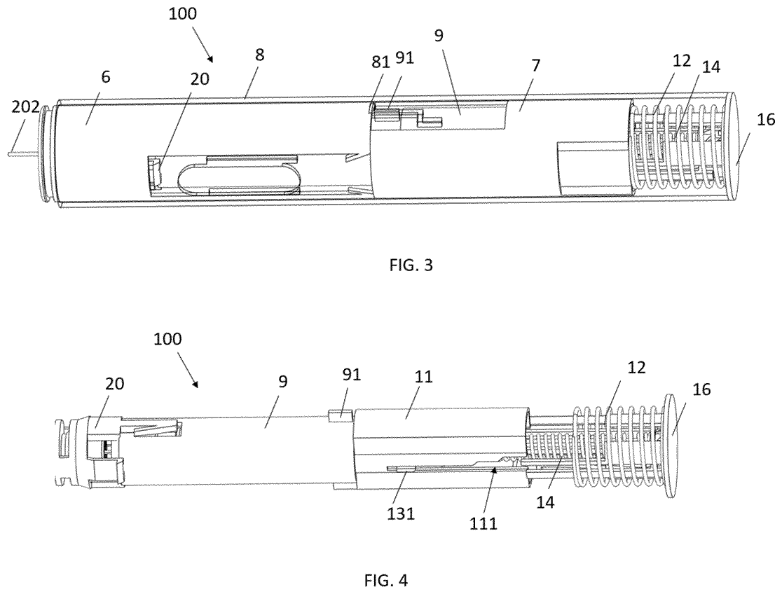

FIG. 3 illustrates a partially transparent perspective view of a medicament delivery device 100 in a state during delivery, according to an embodiment of the present invention, which delivery device preferably is the delivery device of FIGS. 1 and 2. In this state, the needle cover sleeve 6,7 is pushed back into the tubular enclosure 8. This represents the delivery in which the user puts the delivery device 100 with its proximal end onto a dose delivery site, thereby holding the delivery device 100 by its tubular enclosure 8, and pushing the tubular enclosure 8 in the proximal direction. Since the needle cover sleeve 6,7 is slidable arranged in and relative to the tubular enclosure 8, the needle cover sleeve 6,7 moves back into the tubular enclosure 8 against the force exerted by the cartridge driver spring 12. This makes a needle 202 penetrate the user's skin.

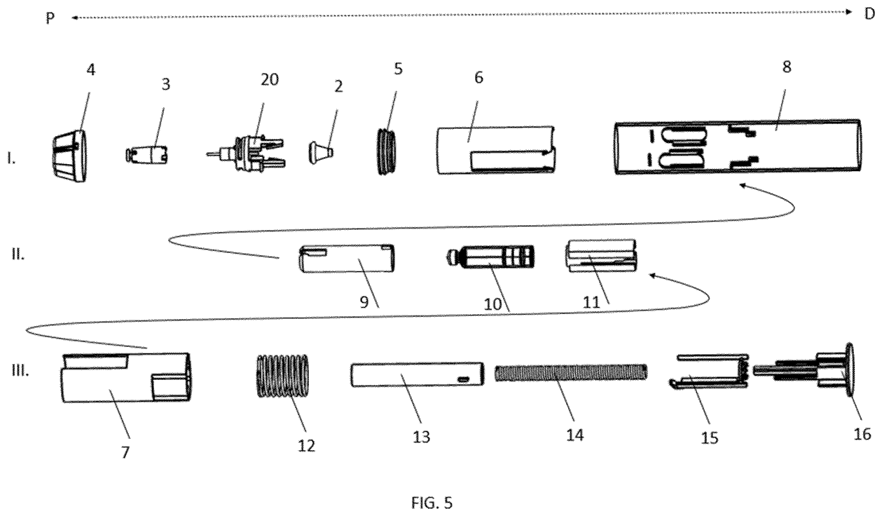

FIG. 4 illustrates a perspective view of the components of the medicament delivery device 100 in a final state of delivery. The tubular enclosure 8 and the needle cover sleeve 6,7 are not illustrated for a better view of the interior components of the delivery device 100. As can be derived from FIG. 4, the cartridge driver 11 includes a mechanical interface portion 111 in form of a recess in a circumferential portion of the tubular cartridge driver 11, extending from its distal end. The mechanical interface portion 111 interacts with an interface member 131 protruding from a circumference of the plunger rod 13. In the present state, the interface member 131 is driven by the plunger rod spring 14 into a position deep into the recess of the cartridge driver 11 representing a position of the plunger rod 13, preferably representing a position in which the plunger rod 13, with its proximal end, deeply dives into the cartridge 10 and has pushed a stopper of the cartridge 10 sufficiently far in the proximal direction to have expelled a majority of the medicament out of the cartridge 10.

FIG. 5 illustrates an exploded view of a medicament delivery device according to an embodiment of the present invention, preferably of the medicament delivery device of FIG. 1. The components of the medicament delivery device are arranged in three rows I., II., and III. The components include the cap 4, a needle cap 3, the needle hub 20, a septum 2, the plug 5, the needle cover sleeve portion 6, the tubular enclosure 8, the cartridge holder 9, the cartridge 10, the cartridge driver 11, the needle cover sleeve portion 7, the cartridge driver spring 12, the plunger rod 13, the plunger rod spring 14, a lock bracket 15 and the rear enclosure 16.

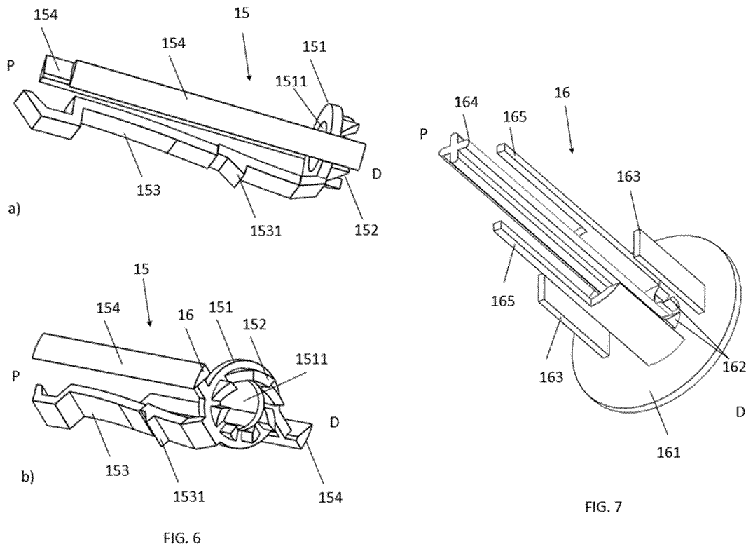

FIG. 6 illustrates the lock bracket 15 as used in a sub-assembly according to an embodiment of the present invention, in two perspectives a) and b). The lock bracket 15 comprises a flange 151 with a central opening 1511, and a mechanical interface portion 152 configured to interact with a corresponding mechanical interface portion 162 of the rear enclosure 16 shown in FIG. 7. Preferably, the interface portion 152 comprises multiple teeth arranged around a circumference, the teeth pointing in the distal direction. Each tooth has a helical-shaped surface. The lock member 15 further comprises a flexible arm 153 extending in the direction of the longitudinal axis along a portion of the plunger rod 13. The flexible arm 153 comprises a distally directed surface 1531. The flexible arm 153 is configured to flex radially inwardly relative to the enclosure 8, 16 from a first position where the distally directed surface 1531 of the flexible arm 153 is engaged with a proximally directed surface of the enclosure 8, 16 to a second position where the distally directed surface 1531 of the flexible arm 153 is disengaged with the proximally directed surface of the enclosure 8, 16.

The lock member 15 further comprises a needle cover sleeve locking member 154, presently comprising two legs protruding in the proximal direction.

FIG. 7 illustrates a perspective view of a rear enclosure 16 as used in a sub-assembly according to an embodiment of the present invention. The rear enclosure 16 comprises a cover 161 at its distal end configured to cover the distal end of the tubular enclosure 8 of any of FIGS. 1 to 3. The rear enclosure 16 further comprises a mechanical interface portion 162 corresponding to the mechanical interface portion 152 of the lock bracket 15.

The corresponding mechanical interface portion 162 also comprises teeth, protruding from the cover 161 in the proximal direction, and configured to interact with the interface portion 152 of the lock bracket 15. Accordingly, it is preferred that the helical surfaces of the teeth of the lock bracket 15 and the rear enclosure 16 matches in slope and inclination. The rear enclosure 16 further comprises a protrusion 163 to interact with the distally directed surface 1531 of the flexible arm 153, presently in form of two legs protruding in the proximal direction. The rear enclosure 16 further comprises a guide rod 164, and an interface member 165 configured to interact with the interface portion 111 of the cartridge driver 11.

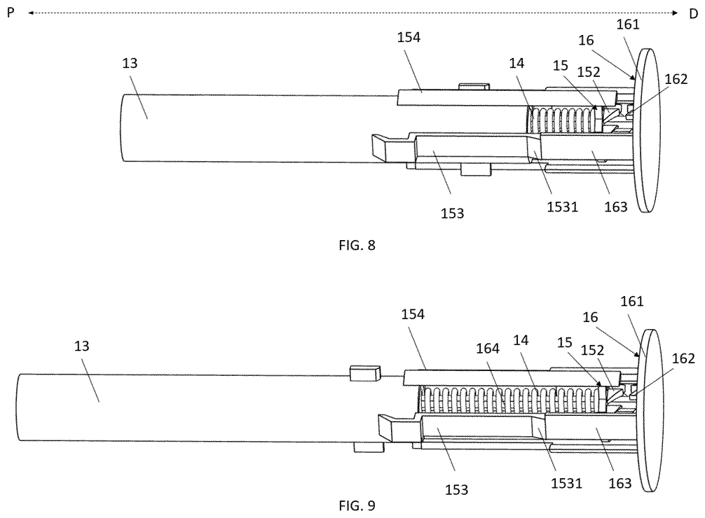

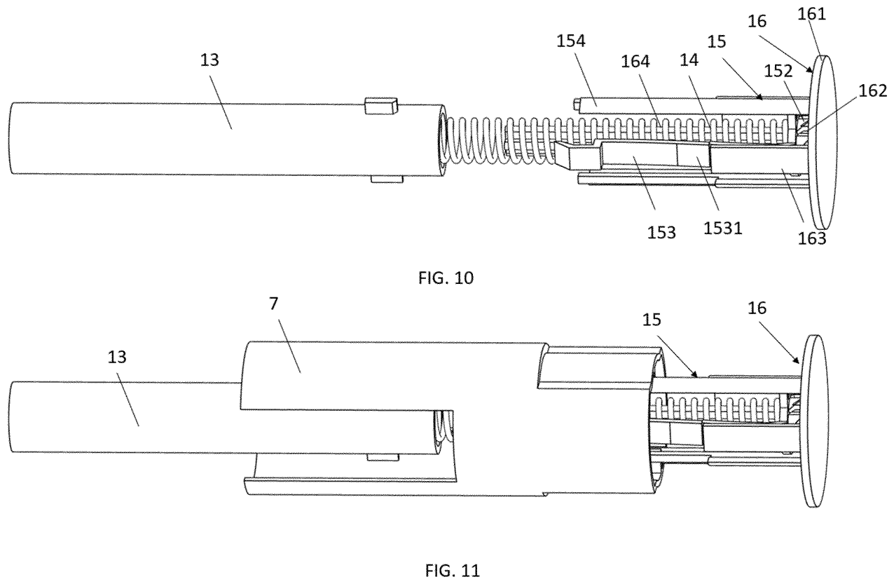

FIG. 8 illustrates a sub-assembly according to an embodiment of the present invention in an initial state. The sub-assembly presently comprises the plunger rod 13, the rear enclosure 16, as shown in FIG. 7, the lock bracket 15 as shown in FIG. 6, and the plunger rod spring 14. The sub-assembly preferably is a sub-assembly as used in the above embodiments of the delivery device 100. The plunger rod 13 is radially lined up with the flexible arm 153 when the plunger rod is in the distal position (as shown in FIG. 9) so that the flexible arm 153 is blocked from moving into the second position and spaced apart from the flexible arm when the plunger rod 13 is in the proximal position (FIG. 10) such that the flexible arm 153 flexes in the second position.

In a preferred example, the plunger rod 13 is tubular-shaped with a front at its proximal end and open at its distal end. The plunger rod spring 14 is arranged in the plunger rod 13 and is biased between the proximal front of the plunger rod 13 and the flange 151 of the lock bracket 15. The guide rod 164 of the rear enclosure 16 reaches through the opening of the flange 151 of the lock bracket 15. The plunger rod spring 14 is arranged on and guided by the guide rod 164. Accordingly, in radial direction the plunger rod spring 14 is arranged between the guide rod 164 and the plunger rod 13. The flexible arm 153 is biased by the outer surface of the plunger rod 13 in the radial direction. By such bias, the distally directed surface 1531 engages with the protrusion 163 of the rear enclosure 16. Accordingly, these two components prevent the lock bracket 15 from moving in the distal direction towards the cover 161 of the rear enclosure 16 despite the force exerted by the plunger rod spring 14. The lock bracket 15 is retained in such a state and such longitudinal position relative to the rear enclosure 16. Accordingly, by these means the lock bracket 15 is arranged distantly from the cover 161 of the rear enclosure 16. This state represents the initial state of the sub-assembly and the corresponding delivery device according to FIG. 1, and also the prepared state in which the delivery device 100 is ready to use.

FIG. 9 illustrates a sub-assembly, and preferably the sub-assembly of FIG. 8 in a state during delivery, according to an embodiment of the present invention. As can be derived from FIG. 9, by a trigger, delivery of the medicament is initiated during which delivery the plunger rod spring 14 is allowed to extend and pushes the plunger rod in the proximal direction, thereby pushing a stopper in the cartridge (not shown) in the proximal direction. This typically causes the medicament to expel from the needle attached to the cartridge at its proximal end. Hence, the plunger rod 13 has moved relative to the rear enclosure 16 and relative to the lock bracket 15 in the proximal direction. The flexible arm 153 of the lock bracket 15 still is radially biased by the plunger rod 13, which does not yet release the engagement between the distally directed surface 1531 and the proximally directed surface of the protrusion 163.

FIG. 10 illustrates a sub-assembly, and preferably the sub-assembly of FIG. 9 in a state of accomplished delivery, according to an embodiment of the present invention. In a transition from the delivery state to the accomplished delivery state, i.e. from the state of FIG. 9 to the state of FIG. 10, the plunger rod 13 is pushed further in the proximal direction by the plunger rod spring 14. As a result, the flexible arm 153 of the lock bracket 15 disengages with the plunger rod 13 and relaxes, thereby moving radially inwards. A

In response to the relaxation of the flexible arm 153, the distally directed surface 1531 disengages with the protrusion 163 of the rear enclosure 16, thereby allowing the plunger rod spring 14, which still is sufficiently biased, to push the lock bracket 15 in the distal direction. As a result, the mechanical interface portion 152 of the lock bracket 15 engages with the corresponding mechanical interface portion 162 of the rear enclosure 16. Such engagement evokes in view of helical surfaces of both interface portions 152,162 a rotation of the lock bracket 15 relative to the rear enclosure 16 in combination with a linear movement in response to the force exerted by the still tensioned plunger rod spring 14. FIG. 10 illustrates a state of the sub-assembly after the engagement of the interface portions 152 and 162.

FIG. 11 illustrates the sub-assembly of FIG. 10 including the needle cover sleeve 7. It is recalled that in the accomplished delivery state, the delivery device looks like in FIG. 3, i.e., the needle cover sleeve 6,7 is retracted into the enclosure 8, and accordingly takes a longitudinal position relative to the rear enclosure 16 as is shown in FIG. 11.

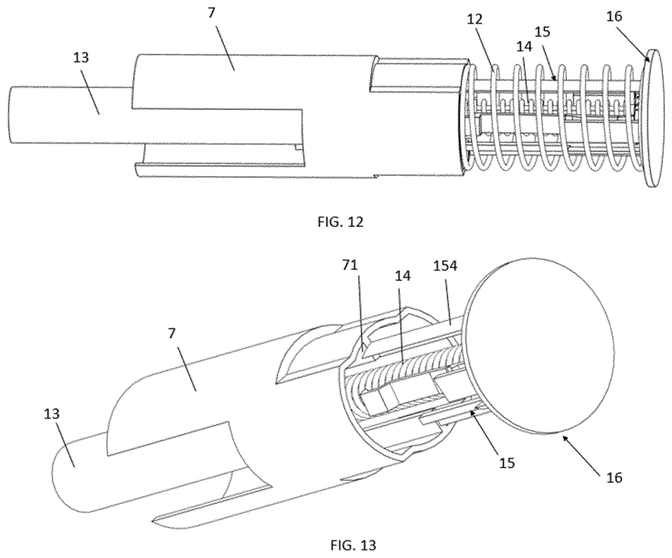

However, in case the user retracts the delivery device from the dose delivery site, the driver spring 12 pushes the needle cover sleeve 6,7 in the proximal direction which resembles the state shown in FIG. 2. In such a state, the needle cover sleeve 6,7 protrudes from the tubular enclosure 8 and protects the needle. This state is illustrated in FIG. 12, now including the drive spring 12. Here, a distance between the needle cover sleeve 6,7 and the platform of the enclosure is increased relative to the delivery accomplished state shown in FIG. 11.

The state illustrated in FIG. 12 is referred to as a locked state given that the following means lock the needle cover sleeve 6,7 in this position relative to the enclosure 8,16. This can best be derived from FIG. 13 which illustrates the sub-assembly of FIG. 12 again in the locked state but from a different perspective. The rotation of the lock bracket 15 described in connection with FIGS. 10 and 11 effects, that the needle cover sleeve locking member 154 rotates from a position in which it was disengaged with the needle cover sleeve 7, into a position in which it is engaged with the needle cover sleeve 7. Presently, the needle cover sleeve 7 comprises a radially inward protrusion 72 serving as a member to interact with the needle cover sleeve locking member 154 in response to the rotation of the lock bracket 15. In this locked state, the needle cover sleeve locking member 154 blocks the needle cover sleeve 6,7 from any translation in the distal direction, and hence from an exposure of the needle by the needle cover sleeve at its proximal end.

The drug delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin generelated peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, Blymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumabpertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g. diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, humanderived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, MiniCHOP, Maxi-CHOP, VR-CAP, Dose-Dense CHOP, EPOCH, Dose-Adjusted EPOCH, REPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DTPACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-17. (canceled)

18. A sub-assembly for a medicament delivery device, extending along a longitudinal axis between a proximal end and a distal end, the sub-assembly comprising:
an enclosure;
a lock bracket arranged in and rotatable relative to the enclosure;
a plunger rod configured to act on a stopper of a cartridge of the medicament delivery device containing a medicament; and
a plunger rod spring arranged between the plunger rod and the lock bracket, wherein the plunger rod spring is configured to move the plunger rod from a distal position to a proximal position relative to the enclosure,
wherein the lock bracket comprises:
a mechanical interface portion configured to engage with a corresponding mechanical interface portion of the enclosure for effecting a rotation of the lock bracket relative to the enclosure and around the longitudinal axis; and
a flexible arm extending in the direction of the longitudinal axis along a portion of the plunger rod, wherein the flexible arm comprises a distally directed surface, wherein the flexible arm is configured to flex radially inwardly relative to the enclosure from a first position where the distally directed surface of the flexible arm is engaged with a proximally directed surface of the enclosure to a second position where the distally directed surface of the flexible arm is disengaged with the proximally directed surface of the enclosure,
wherein the plunger rod is radially lined up with the flexible arm when the plunger rod is in the distal position so that the flexible arm is blocked from moving into the second position and spaced apart from the flexible arm when the plunger rod is in the proximal position such that the flexible arm flexes in the second position, such that the plunger rod spring pushing the lock bracket in the distal direction to interact with the mechanical interface portions and effecting the rotation of the lock bracket relative to the enclosure.

19. The sub-assembly according to claim 18, wherein the mechanical interface portion is arranged at the distal end of the lock bracket and comprises a flange with at least one tooth protruding in the distal direction.

20. The sub-assembly according to claim 19, wherein each tooth of the mechanical interface portion comprises a helical surface configured to effect the rotation of the lock bracket when interacting with the corresponding mechanical interface portion of the enclosure, preferably wherein multiple teeth are arranged circumferentially on the mechanical interface portion.

21. The sub-assembly according to claim 18, wherein the enclosure comprises a tubular enclosure including an opening at its distal end, and a rear enclosure attached to the tubular portion and comprising a cover for covering the opening.

22. The sub-assembly according to claim 21, wherein the corresponding mechanical interface is provided at the rear enclosure and comprises at least one tooth protruding from the cover in the proximal direction.

23. The sub-assembly according to claim 22, wherein each tooth of the corresponding mechanical interface portion comprises a helical surface configured to affect the rotation of the lock bracket when interacting with the mechanical interface portion of the lock bracket, preferably wherein multiple teeth are arranged circumferentially on the cover.

24. The sub-assembly according to claim 18,
wherein the plunger rod is of tubular-shaped and comprises an outer cylindrical surface,
wherein, when the plunger rod is in the distal position, the flexible arm engages with the outer surface of the plunger rod and is radially biased by the plunger rod, and
wherein, when the plunger rod is in the proximal position, the flexible is disengaged with the plunger rod.

25. The sub-assembly according to claim 24, wherein, when the plunger rod is in the proximal position, the flexible arm is in a relaxed, unbiased state.

26. The sub-assembly according to claim 18, wherein the flexible arm comprises a radially protruding ledge, and wherein the radially protruding ledge comprises the distally directed surface.

27. The sub-assembly according to claim 21, wherein the enclosure comprises a protrusion protruding from the cover of the rear enclosure in the proximal direction, and wherein the protrusion comprises the proximally directed surface of the enclosure.

28. The sub-assembly according to claim 21,
wherein the rear enclosure comprises a guide rod extending in the proximal direction,
wherein the plunger rod spring is radially arranged between the guide rod and the plunger rod, and
wherein the lock bracket is slidable and arranged on the guide rod against the force of the plunger rod spring.

29. The sub-assembly according to claim 28, wherein the cover, the guide rod, the corresponding interface portion and the protrusion of the rear enclosure are made integral.

30. The sub-assembly according to claim 18,
wherein the plunger rod is tubular shaped and comprises a front at its proximal end,
wherein the lock bracket comprises a flange arranged at its distal end, and
wherein the plunger rod spring reaches into the tubular plunger rod and is biased between the front of the plunger rod and the flange of the lock bracket.

31. The sub-assembly according to claim 18, further comprising:
an indicator for one or more visually, audibly and tactile indicating an extended state of the plunger rod.

32. A medicament delivery device, comprising:
the sub-assembly according to claim 18;
a needle to expel medicament from a cartridge of the medicament delivery device; and
a needle cover sleeve arranged in and slidable relative to the enclosure between a needle protecting position and a needle exposing position.

33. The medicament delivery device of claim 32,
wherein the lock bracket comprises a needle cover sleeve locking member configured to engage with the needle cover sleeve in response to the rotation of the lock bracket, in which engaged position a transition of the needle cover sleeve from the needle protecting position into the needle exposing position is locked,
preferably wherein the needle cover sleeve locking member comprises one or more legs extending in the proximal direction.

34. The medicament delivery device according to claim 33, wherein the needle cover sleeve comprises a protrusion arranged to engage with the needle cover sleeve locking member in response to the rotation of the lock bracket,
preferably wherein the needle cover sleeve locking member comprises a transversal locking surface configured to engage with a transversal locking surface of the protrusion of the needle cover sleeve.
